SEQUENCE LISTING (12 sequences)

SEQ ID NO: 1 - protein, 72 aa, synthetic construct
  REGION 1..72: note = MCD0
  VARIANT 8: note = X is R
  VARIANT 19: note = X is H or K
  VARIANT 21: note = X is H or K
  VARIANT 65: note = X is H or K
APIANELXCQCLQTMAGIXLXNIQSLCVLPSGPHCTQTEVIATLKNGREACLDPEAPLVQKIVQXMLKGVPK

SEQ ID NO: 2 - protein, 72 aa, synthetic construct
  REGION 1..72: note = HCD0
  VARIANT 6: note = X is R
  VARIANT 18: note = X is H or K
  VARIANT 20: note = X is H or K
  VARIANT 64: note = X is H or K
SAKELXCQCIKTYSKPFXPXFIKELCVIESGPHCANTEIIVKLSDGRELCLDPKENWVQRVVEXFLKRAENS

SEQ ID NO: 3 - protein, 72 aa, synthetic construct
  REGION 1..72: note = MCD1
  VARIANT 19: note = X is H or K
  VARIANT 21: note = X is H or K
  VARIANT 65: note = X is H or K
APIANELACQCLQTMAGIXLXNIQSLCVLPSGPHCTQTEVIATLKNGREACLDPEAPLVQKIVQXMLKGVPK

SEQ ID NO: 4 - protein, 72 aa, synthetic construct
  REGION 1..72: note = HCD1
  VARIANT 18: note = X is H or K
  VARIANT 20: note = X is H or K
  VARIANT 64: note = X is H or K
SAKELACQCIKTYSKPFXPXFIKELCVIESGPHCANTEIIVKLSDGRELCLDPKENWVQRVVEXFLKRAENS

SEQ ID NO: 5 - protein, 72 aa, synthetic construct
  REGION 1..72: note = MCD2
  VARIANT 21: note = X is K
  VARIANT 65: note = X is K
APIANELACQCLQTMAGIRLXNIQSLCVLPSGPHCTQTEVIATLKNGREACLDPEAPLVQKIVQXMLKGVPK

SEQ ID NO: 6 - protein, 72 aa, synthetic construct
  REGION 1..72: note = HCD2
  VARIANT 20: note = X is K
  VARIANT 64: note = X is K
SAKELACQCIKTYSKPFRPXFIKELCVIESGPHCANTEIIVKLSDGRELCLDPKENWVQRVVEXFLKRAENS

SEQ ID NO: 7 - protein, 72 aa, synthetic construct
  REGION 1..72: note = MCD2
  VARIANT 19: note = X is H or K
  VARIANT 65: note = X is H or K
APIANELACQCLQTMAGIXLRNIQSLCVLPSGPHCTQTEVIATLKNGREACLDPEAPLVQKIVQXMLKGVPK

SEQ ID NO: 8 - protein, 72 aa, synthetic construct
  REGION 1..72: note = HCD2
  VARIANT 18: note = X is H or K
  VARIANT 64: note = X is H or K
SAKELACQCIKTYSKPFXPRFIKELCVIESGPHCANTEIIVKLSDGRELCLDPKENWVQRVVEXFLKRAENS

SEQ ID NO: 9 - protein, 72 aa, synthetic construct
  REGION 1..72: note = MCD2
  VARIANT 19: note = X is H or K
  VARIANT 21: note = X is H or K
APIANELACQCLQTMAGIXLXNIQSLCVLPSGPHCTQTEVIATLKNGREACLDPEAPLVQKIVQRMLKGVPK

SEQ ID NO: 10 - protein, 72 aa, synthetic construct
  REGION 1..72: note = HCD2
  VARIANT 18: note = X is H or K
  VARIANT 20: note = X is H or K
SAKELACQCIKTYSKPFXPXFIKELCVIESGPHCANTEIIVKLSDGRELCLDPKENWVQRVVERFLKRAENS

SEQ ID NO: 11 - protein, 72 aa, synthetic construct
  REGION 1..72: note = mCxcl1
APIANELRCQCLQTMAGIHLKNIQSLKVLPSGPHCTQTEVIATLKNGREACLDPEAPLVQKIVQKMLKGVPK

SEQ ID NO: 12 - protein, 72 aa, synthetic construct
  REGION 1..72: note = hCXCL8
SAKELRCQCIKTYSKPFHPKFIKELRVIESGPHCANTEIIVKLSDGRELCLDPKENWVQRVVEKFLKRAENS

(19) United States
(12) Patent Application Publication
(10) Pub. No.: US 2026/0022152 A1
(43) Pub. Date: Jan. 22, 2026

(54) CHEMOKINE BIOLOGICS FOR TREATING INFLAMMATION-RELATED DISEASES AND DISORDERS

(71) Applicant: Board of Regents, The University of Texas System, Austin, TX (US)

(72) Inventors: Krishna Rajarathnam, Galveston, TX (US); Ashok Chopra, Galveston, TX (US); Sara Dann, Galveston, TX (US)

(21) Appl. No.: 19/273,455

(22) Filed: Jul. 18, 2025

Related U.S. Application Data
(60) Provisional application No. 63/673,267, filed on Jul. 19, 2024.

(51) Int. Cl. C07K 14/52; A61K 38/00; A61P 29/00

(52) U.S. Cl. CPC C07K 14/522; A61P 29/00; A61K 38/00

(57) ABSTRACT

Provided herein are compositions and methods for treating one or more symptoms of dysregulated inflammation in an organ of a subject in need thereof, the method comprising: administering to the subject a composition comprising a chemokine biologic that has a higher affinity for glycosaminoglycans (GAGs) compared to wild-type chemokine and that modulates an interaction between native chemokine and GAGs in an amount effective to treat one or more symptoms of dysregulated inflammation, and a pharmaceutically acceptable carrier.

### CROSS-REFERENCE TO RELATED APPLICATIONS

This application claims priority to U.S. Provisional Application Ser. No. 63/673,267, filed Jul. 19, 2024 entitled “Chemokine Decoys for Treating Hyperinflammation Related Diseases and Disorders”, which is hereby incorporated by reference in its entirety.

### TECHNICAL FIELD

The present disclosure relates in general to the field of dysregulated inflammation and neutrophil-driven inflammatory responses, and more particularly, to novel chemokine biologics for treating inflammation-related diseases and disorders.

### STATEMENT OF FEDERALLY FUNDED RESEARCH

Not applicable.

### INCORPORATION-BY-REFERENCE OF MATERIALS FILED ON COMPACT DISC

The application includes a Sequence Listing which has been submitted in .XML format and is hereby incorporated by reference in its entirety. Said XML copy, created on Jul. 30, 2025, is named UTMB 1078.xml and is 26,176 bytes in size. The sequence listing contained in this .XML file is part of the specification and is hereby incorporated by reference herein in its entirety.

### BACKGROUND

Without limiting the scope of the invention, its background is described in connection with inflammation-related diseases and disorders.

One such treatment is taught in U.S. Patent Publication No. 20230165904, filed by Itescu, entitled, “Method For Treating Hyperinflammation Using Mesenchymal Lineage Precursor Or Stem Cells”. This applicant is said to teach a method of treating or preventing hyperinflammation in a subject in need thereof, the method comprising administering to the subject a composition comprising mesenchymal lineage precursor stem cells (MLPSCs).

Another such treatment is taught in U.S. Patent Publication No. 20180185443, filed by Cotereau, entitled, “Kit For Treating Sepsis and/or Any Systemic (SIRS) Or Damaging Cellular Hyperinflammation”. This applicant is said to teach a kit including at least one antioxidant selenoprotein and at least one composition including at least one oxidant selenocompound, including administering effective and cytotoxic doses of selenocompounds, allowing the inhibition of the hyperactivation of phagocytes and in particular of circulating immature neutrophils and directly and indirectly protecting endothelial cells, in particular for the treatment of sepsis, SIRS and leukemia.

Despite these advances, a need remains for novel therapeutics that specifically correct dysregulated inflammation, whether by reducing or inhibiting hyperinflammatory or augmenting impaired hypoinflammatory responses.

### SUMMARY

As embodied and broadly described herein, an aspect of the present disclosure relates to a method for treating one or more symptoms of dysregulated inflammation in an organ of a subject in need thereof, the method comprising: administering to the subject a composition comprising a chemokine biologic, e.g., a decoy, that has a higher affinity for glycosaminoglycans (GAGs) compared to an endogenous or wild-type chemokine and modifies, e.g., reduces, interactions between native chemokine and GAGs in an amount effective to treat one or more symptoms of dysregulated inflammation, and a pharmaceutically acceptable carrier. In one aspect, the dysregulated inflammation is systemic inflammation involving sepsis or septic shock, multiple organs, tissues, and/or cell types of the body. In another aspect, the chemokine biologic forms a locked dimer. In another aspect, the chemokine biologic is selected from at least one of SEQ ID NOS: 1-10. In another aspect, the severe inflammation is caused by inflammatory bowel disease, peritonitis, bacterial infections, and cancer. In another aspect, the bacterial infection with Salmonella, Klebsiella, Yersinia, or Clostridium strains. In another aspect, the dysregulated inflammation is characterized by cytokine storm. In another aspect, hyperinflammation is associated with acute lung injury (ALI) and/or acute respiratory distress syndrome (ARDS) associated with or arising from ventilator use, viral infection, sepsis, or systemic bacterial infections. In another aspect, the chemokine biologic redirects a neutrophil's dysregulated inflammatory response. In another aspect, the composition is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration. In another aspect, the composition is formulated for delivery enterally or to a mucosal surface. In another aspect, the composition is administered via intravenous, subcutaneous, or intramuscular route. In another aspect, the composition is administered prior to, in conjunction, subsequent to, or in alternation with treatment with one or more additional therapies or procedures selected from the group consisting of antimicrobials, surfactant, and corticosteroids. In another aspect, the composition is in an amount between about 0.1 and about 40 mg/kg body weight of the subject. In another aspect, the composition is in an amount between about 2 and about 8 mg/kg body weight of the subject, and wherein the composition is administered by subcutaneous injection. In another aspect, the composition is administered in an amount effective to regulate, e.g., reduce, the activity of one or more pro-inflammatory chemokines selected from the group consisting of CXCL1, CXCL2, CXCL3, CXCL5, CXCL6, CXCL7, and CXCL8. In another aspect, the composition is administered in an amount effective to regulate the activity of one or more pro-inflammatory cytokines selected from IL-6, TNF-α, IL-12, IL-1α, and IL-18, and other inflammation related proteins, such as C-reactive protein (CRP) and ferritin. In another aspect, the one or more pro-inflammatory cells are neutrophils.

As embodied and broadly described herein, an aspect of the present disclosure relates to a peptide comprising a chemokine biologic that has a higher affinity for glycosaminoglycans (GAGs) compared to wild-type chemokines and that regulates binding to GAGs in an amount effective to treat one or more symptoms of dysregulated inflammation. In one aspect, the chemokine biologic forms a locked dimer. In another aspect, the chemokine biologic is a chemokine variant or mutant selected from at least one of SEQ ID NOS: 1-10. In another aspect, the biologic further comprises a pharmaceutically acceptable carrier. In another aspect, the biologic is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration. In another aspect, the biologic is formulated for delivery enterally or to a mucosal surface. In another aspect, the biologic is in an amount between about 0.1 and about 40 mg/kg body weight of the subject. In another aspect, the biologic is in an amount between about 2 and about 8 mg/kg body weight of the subject, and wherein the chemokine biologic is administered by subcutaneous injection.

As embodied and broadly described herein, an aspect of the present disclosure relates to a method for treating one or more symptoms of severe inflammation in an organ of a subject in need thereof. The method comprising: identifying a subject in need of treatment for dysregulated inflammation triggered by neutrophils; and administering to the subject a composition comprising a chemokine biologic that has a higher affinity for GAGs compared to endogenous wild-type chemokines, wherein the biologic regulates GAG binding in an amount effective to treat one or more symptoms of dysregulated inflammation.

As embodied and broadly described herein, an aspect of the present disclosure relates to a method for treating one or more symptoms of dysregulated inflammation in an organ of a subject in need thereof, the method comprising: administering to the subject a biologic that has a higher affinity for GAGs compared to endogenous wild-type chemokine and that regulates binding to GAGs in an amount effective to treat one or more symptoms of dysregulated inflammation, and a pharmaceutically acceptable carrier. In one aspect, the biologic is administered in an amount effective to regulate the activity of one or more pro-inflammatory chemokines selected from the group consisting of CXCL8, CXCL1, CXCL2, CXCL3, CXCL6, CXCL5, and CXCL7. In another aspect, the biologic is administered in an amount effective to regulate the levels of one or more pro-inflammatory cytokines selected from IL-6, TNF-α, IL-12, IL-1α, and IL-18, and inflammation-related proteins C-reactive protein (CRP) and ferritin. In another aspect, the one or more pro-inflammatory cells are neutrophils.

### BRIEF DESCRIPTION OF THE FIGURES

For a more complete understanding of the features and advantages of the present disclosure, reference is now made to the detailed description of the disclosure along with the accompanying figures and in which:

FIGS. 1A and 1B are schematics showing (FIG. 1A) neutrophil migration up the chemokine gradient from low to high concentration; and (FIG. 1B) how a biologic can correct suboptimal chemokine gradients and redirect detrimental neutrophil responses into protective ones.

FIG. 2 shows a schematic highlighting the basic residues in the chemokine human CXCL8 involved in binding to GAGs.

FIG. 3 is a schematic of the chemokine CD1 biologic created on the CD0 background. Arg mutation to Ala is highlighted. The CD0 biologic was created by introducing a disulfide across the dimer interface (in circles).

FIG. 4 is a graph showing a survival plot of 7 to 9 weeks old (n=5) mice (C57BL/6 from Jackson Labs) intraperitoneally (i.p.) challenged with a lethal dose of Salmonella Typhimurium strain 14028 (1×10⁶ cfu) and treated with chemokine biologic (CD0). Untreated mice serve as a control. The biologic (10 μg in 100 μl of PBS buffer) was administered via the intraperitoneal route 1 hour before the challenge. All control (untreated) mice die by day 4, whereas a significant number of biologic-treated mice survive. Data were analyzed using log-ranked Mantel Cox test. **p<0.01.

FIG. 5 is a graph showing a survival plot of 7 to 9 weeks old mice (C57BL/6 from Jackson Labs) (n=13 to 17) intraperitoneally (i.p.) challenged with a lethal dose of Klebisella pneumoniae strain 43816 (1×10³ cfu) and treated with chemokine biologic (CD0). Untreated mice serve as a control. The biologic (10 μg in 100 μl of PBS buffer) was administered via the intraperitoneal route one hour before the challenge. All untreated mice die by day 3, whereas a significant number of biologic-treated mice survive. Data were analyzed using log-ranked Mantel Cox test. **p<0.01.

FIG. 6 is a graph showing a survival plot of 7 to 9 weeks old (n=5) mice (C57BL/6 from Jackson Labs) intranasally (i.n.) challenged with a lethal dose of Klebisella pneumoniae strain 43816 (1×10⁵ cfu) and treated with chemokine biologic (CD0). Untreated mice serve as a control. The biologic (10 μg in 20 μl of PBS buffer) was administered via the intranasal route 1 hour before the challenge. All control (untreated) mice die by day 4, whereas a significant number of biologic-treated mice survive. Data were analyzed using log-ranked Mantel Cox test. *p<0.05.

FIG. 7 is a graph showing a survival plot of 7 to 9 weeks old Swiss Albino mice (n=5) intranasally (i.n.) challenged with a lethal dose of Yersinia Pestis strain CO92 luc2 (12 LD50) and treated with chemokine biologic (CD0). The biologic (10 μg in 20 μl of PBS buffer) was administered via the intranasal route 1 hour before the challenge. All control (PBS) mice die by day 4. In this experiment, the biologic by itself is not protective and the antibiotic levofloxin imparts some protection. However, the CD0 biologic synergistically provides better protection in combination with the antibiotic levofloxin compared to antibiotic alone. Data were analyzed using log-ranked Mantel Cox test. *p<0.01 (compared to untreated mice).

FIGS. 8A and 8B are graphs showing survival plots of 7 to 9 weeks old (n=5) mice (C57BL/6 from Jackson Labs) intraperitoneally (i.p.) challenged with a lethal LPS dose from E. coli 0111.B4 and treated with chemokine biologic CD1 (FIG. 8A) and CD2 (FIG. 8B), respectively. The biologic (10 μg in 100 μl of PBS buffer) was administered via the intraperitoneal route 1 hour before the challenge. All untreated mice serve as a control and die by day 2, whereas a significant number of biologic-treated mice survive. Data were analyzed using log-ranked Mantel Cox test. **p<0.01.

FIG. 9 is a graph showing a survival plot of 7 to 9 weeks old (n=5) mice (C57BL/6 from Jackson Labs) with Clostrodium difficile strain VPI 10463 (1×10⁴ spores) and treated with chemokine biologic CD2 6 hours before the challenge. All control (PBS) mice die by day 12, whereas a significant number of biologic-treated mice survive. Data were analyzed using log-ranked Mantel Cox test. **p<0.01, *p<0.05.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

As used herein, the term “biologic” refers to a composition that is or may be produced by recombinant DNA technologies, peptide synthesis, chemical synthesis, and combinations thereof, that has a desired biological activity. The biologic can be, for example, a protein, peptide, glycoprotein, a mixture of proteins or peptides, a mixture of glycoproteins, or a derivatized or modified form of any of the foregoing. In certain examples, the biologic is a drug used for treatment of diseases and/or medical conditions. Examples of biologic drugs include, without limitation, native or engineered protein, peptide, glycoprotein, a mixture of proteins or peptides, a mixture of glycoproteins, or a derivatized or modified form and can also be conjugated directly or indirectly (e.g., via a side chain or linker) to a drug of interest or a molecule that modifies the binding of the protein or peptide. In certain examples, the biologic is a decoy. In certain embodiments, the biologic can be used in an assay, e.g., to detect the presence of binding target for the biologic. The biologics may also be used as medical imaging agents, such as peptides or proteins that are labelled with agents that provide a detectable signal that facilitates imaging such as fluorescent markers, dyes, radionuclides, and the like.

As used herein, the terms “treating”, “treat” or “treatment” include reducing or eliminating one or more symptoms of hyperinflammation and hypoinflammation. In one example, the treatment includes administering one of several biologics, e.g., a decoy, that regulate binding of native (wild-type) chemokines to glycosaminoglycans (GAGs). In one example, the one or more biologics reduce or eliminate the binding of native chemokines to GAGs.

As used herein, the terms “prevent” or “preventing” includes administering one of several chemokine biologics to prevent, stop, or inhibit the development of at least one symptom of dysregulated inflammation. In one example, the treatment includes administering one of several biologics that prevent or reduce binding of native chemokines to GAGs.

As used herein, the terms “reducing” or “inhibiting” refer to reducing or eliminating disease progression or disease complication in a subject by inhibiting the initiation or progression of hyperinflammation. In one example, the treatment includes administering one of several biologics that regulate binding of native (wild-type) chemokines to GAGs.

As used herein, the terms “hyperinflammation” and “hypoinflammation” refer to severe and ongoing dysregulated inflammatory process in the body. For example, dysregulated inflammation can refer to severe and ongoing hyperinflammatory process in the blood stream, such as sepsis, or in the airways/lungs, kidney, and/or liver. In this way, dysregulated inflammation can affect multiple organs in the body and their vasculature. In one example, the dysregulated inflammation is triggered by a bacterial, viral, or parasitic infection. Hyperinflammation is associated with a cytokine storm or cytokine release syndrome (CRS). In an example, the cytokine storm or CRS involves significant release of inflammatory cytokines including chemokines.

In one example, hyperinflammation is associated with elevated chemokines and/or cytokines. Hyperinflammation can be measured by detecting certain biomarkers. For example, ferritin levels may be greater than 1000 ng/ml.

In hyperinflammation associated with bacterial infection, it can be measured as the subject having elevated circulating CRP levels, e.g., circulating CRP levels greater than 100 mg/dl, greater than 120 mg/dl, greater than 150 mg/dl, or having circulating CRP levels between 90 mg/dl and 300 mg/dl.

Hyperinflammation can also be associated with elevated triglycerides or decreased fibrinogen. For example, subjects treated according to the present disclosure can have triglyceride levels >1.5, >2, >3, >4 mg/L, between 1.5 and 5 mg/L.

Subjects treated according to the present disclosure may have symptoms indicative of dysregulated inflammation. Exemplary symptoms of hyperinflammation may include one or more of the following: fatigue, trouble breathing, shortness of breath, inability or decreased ability to exercise, coughing with or without blood or mucus, pain when breathing in or out, wheezing, chest tightness, unexplained weight loss, and musculoskeletal pain.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have structures that are different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “polypeptide,” “peptide” and “protein” are used interchangeably herein to refer to a polymer of amino acid residues, wherein the polymer may, in certain aspects, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular aspects, a pharmaceutical composition is suitable for specific or non-specific immunotherapy, or is a vaccine composition.

As used herein, the word “expression” or “expressed” in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

As used herein, the phrase “conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine(S), Threonine (T); and (8) Cysteine (C), Methionine (M) (see, e.g., Creighton, Proteins (1984)).

As used herein, the phrase “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or 100% identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. The present invention includes peptides of any one of SEQ ID NOS: 1 to 6, that may have 85%, 90%, 91%, 92%, 93%, 94%, 95%, 96%, 97%, 98%, 99%, or 100% identity.

As used herein, the term “effective amount” or “effective dose” refers to that amount of the one or more chemokine biologics, e.g., decoys, to prevent and/or correct dysregulated inflammation. The hyperinflammation and hypoinflammation may be triggered by neutrophils. An effective dose may refer to the amount of one or more chemokine biologics sufficient to correct hyperinflammation and hypoinflammation. Further, an effective dose is the amount with respect to one or more chemokine biologics of the invention alone, or in combination with other therapies, that provides a therapeutic benefit in the treatment or management of inflammation. A reduction of a symptom or symptoms (and grammatical equivalents of this phrase) of dysregulated inflammation refers to decreasing of the severity or frequency of the symptom(s), or elimination of the dysregulated inflammation symptom(s). A prophylactically effective amount refers to an amount of the one or more chemokine biologics that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, inflammation and/or hyperinflammation. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1 or 2, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “administering” refers to the administration of the one or more chemokine biologics including, e.g., by intravenous, intraperitoneal, intramuscular, intralesional, intrathecal, intranasal, pulmonary, oral, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols.

Neutrophils, first responders at the site of infection, play a central role in destroying pathogens, and employ a formidable arsenal of cytotoxic proteases, reactive oxygen species, and extracellular traps. Besides eliminating pathogens, they are also instrumental in initiating repair and restoring homeostasis. Therefore, neutrophil function must be highly regulated, as an imbalanced response-uncontrolled or impaired-precipitates significant tissue damage and exacerbates the disease process (1, 2). Chemokines, released at the insult site by resident cells, orchestrate neutrophil recruitment and determine its phenotype. Chemokine function is determined by three characteristic properties: activating the chemokine CXCR1 and CXCR2 receptors, existing reversibly as a monomer and a dimer, and binding to glycosaminoglycans (GAGs) (3-4). Chemokine monomer and dimer binding to GAGs, which are sulfated polysaccharides, determine soluble chemotactic and GAG-bound haptotactic chemokine gradients. These gradients and receptor signaling, acting in concert, determine neutrophil response at the insult site (5-14).

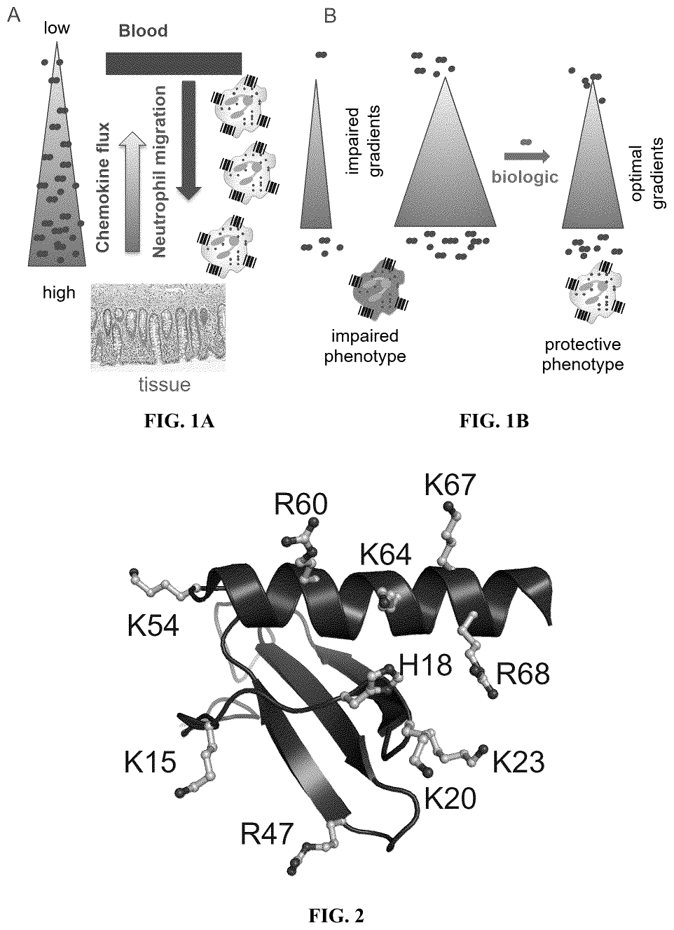

An optimal balance of the chemokine between free and GAG-bound monomer and dimer forms is critical for successful neutrophil response. Under conditions of duress, an imbalance in this equilibrium results in a dysregulated neutrophil response and contributes to the disease etiology. An imbalance could occur due to lower or higher chemokine expression levels. The present inventors engineered novel chemokine biologics that can tune the activity of the endogenous chemokines. By exploiting chemokine structural and functional properties, they have designed a series of biologics that have varying but always higher GAG affinities than endogenous chemokines and varying receptor signaling activities. These biologics will differ in their ability in enforcing a new balance between free and bound chemokine forms, reshaping gradients, and modulating receptor signaling (FIGS. 1A, 1B). Therefore, these biologics could function as therapeutics for a range of neutrophil activation phenotypes, from excessive to impaired, in a context-dependent manner.

The present inventors designed chemokine biologics that bind GAGs with higher affinity compared to native chemokines. The strategy involved enhancing H-bonding and ionic interactions by exploiting the observations that GAG affinity can be increased by substituting an arginine (Arg) for a lysine (Lys) or a histidine (His) and that chemokine dimers bind GAGs with higher affinity than do monomers. The inventors designed and characterized biologics of mouse chemokine Cxcl1 on a locked dimer template that also carries a mutation that disables receptor signaling.

The first Cxcl1 biologic variants were found to bind GAGs with as much as 20-fold higher affinity, and it is shown herein that some of the biologics were able to attenuate disease progression and increase survival in a lethal peritonitis mouse model. The chemokine biologics disclosed herein functioning as therapeutics for neutrophil-related pathology is novel, and uses a strategy for engineering biologics by enhancing H-bonding and ionic interactions by exploiting the chemical properties and side chain structures of basic residues.

Protein therapeutics such as chemokine biologics offer several advantages, as can be noted in the major rise of protein-based biologics being approved by the FDA. These include minimal or no off-target effects, ease of delivery, close to 100% bioavailability, stability, and tunable duration of action. Chemokine structure-based biologics are stable, small, cost effective, do not contain post-translational modifications, easily purified, easily meet GMP specifications, and have good pharmacokinetic and pharmacodynamic properties. The chemokine biologics disclosed herein meet all of the requirements for use in humans. High-affinity GAG-binding chemokine biologics can work as a standalone or in a combination regimen for treating neutrophil-mediated diseases including inflammatory bowel disease, peritonitis, bacterial infections, and cancer. The chemokine biologics of the present disclosure function as a pan-inhibitor by regulating binding of other GAG-binding proteins that promote inflammation such as those involved in the coagulation cascade.

The present disclosure includes biologics using human CXCL8 (also known as interleukin-8, IL-8) as the template. Rational design of biologics requires knowledge of the structural basis and molecular mechanisms by which chemokine binds GAGs. The inventors used solution NMR spectroscopy, docking, and molecular dynamics (MD) studies. As CXCL8 reversibly exists as a monomer and dimer, the inventors also characterized the binding of GAG heparin to CXCL8 at concentrations where signals corresponding to both the monomer and dimer can be observed in the NMR spectrum (15, 16). Upon adding heparin, the peaks corresponding to the monomer disappear indicating the dimer binds GAG with higher affinity. By way of explanation, but not a limitation of the present disclosure, higher dimer affinity can be attributed to differences in structural dynamics between the monomer and dimer in the free and GAG-bound forms.

FIGS. 1A and 1B are schematics showing (FIG. 1A) neutrophil migration up the chemokine gradient from low to high concentration; and (FIG. 1B) how a biologic can correct suboptimal chemokine gradients and redirect detrimental neutrophil responses into protective ones.

FIG. 2 shows a schematic highlighting the basic residues in the chemokine human CXCL8 involved in binding to GAGs.

GAGs are acidic and CXCL8 is basic, and so binding and recognition is driven by H-bonding and ionic interactions (15). NMR studies identified that GAG binding is mediated by residues Lys15 (K15), His18 (H18) and Lys20 (K₂O) from the N-loop; Arg47 (R47) from the 40 s turn; and Arg60 (R60), Lys64 (K64), Lys67 (K67), and Arg68 (R68) from the C-terminal α-helix (FIG. 2). The inventors carried out High Ambiguity Driven protein-protein DOCKing (HADDOCK)-based docking to understand how the topology and distribution of these basic residues and acidic groups in GAG determine complex formation. Structural models indicate that basic residues form a contiguous surface spanning one face of the protein within one monomer of the dimer and that GAG adopts a specific geometry in the bound complex (FIG. 2). MD measurements indicate that only a subset of interface residues contribute to most of the binding affinity and that binding interactions of several GAG-interface residues are less than optimal. These observations were critical in designing high-affinity GAG-binding chemokine biologics that regulate, e.g., disrupt, native chemokine-GAG interactions and thereby tune neutrophil response.

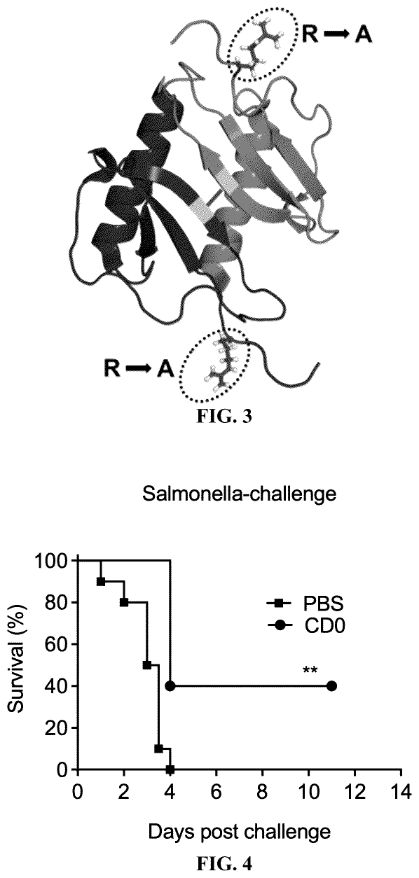

FIG. 3 is a schematic of the chemokine CD1 biologic created on the CD0 background. Arg mutation to A1a is highlighted. The CD0 biologic was created by introducing a disulfide across the dimer interface (in circles).

FIG. 4 is a graph showing a survival plot of 7 to 9 weeks old (n=5) mice (C57BL/6 from Jackson Labs) intraperitoneally (i.p.) challenged with a lethal dose of Salmonella Typhimurium strain 14028 (1×10⁶ cfu) and treated with chemokine biologic (CD0). Untreated mice serve as a control. The biologic (10 μg in 100 μl of PBS buffer) was administered via the intraperitoneal route 1 hour before the challenge. All control (untreated) mice die by day 4, whereas a significant number of biologic-treated mice survive. Data were analyzed using log-ranked Mantel Cox test. **p<0.01.

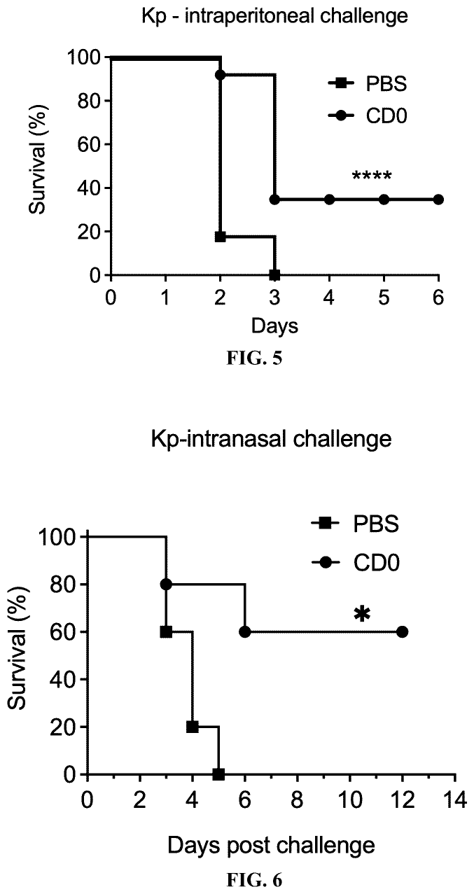

FIG. 5 is a graph showing a survival plot of 7 to 9 weeks old mice (C57BL/6 from Jackson Labs) (n=13 to 17) intraperitoneally (i.p.) challenged with a lethal dose of Klebisella pneumoniae strain 43816 (1×10³ cfu) and treated with chemokine biologic (CD0). Untreated mice serve as a control. The biologic (10 μg in 100 μl of PBS buffer) was administered via the intraperitoneal route one hour before the challenge. All untreated mice die by day 3, whereas a significant number of biologic-treated mice survive. Data were analyzed using log-ranked Mantel Cox test. **p<0.01.

FIG. 6 is a graph showing a survival plot of 7 to 9 weeks old (n=5) mice (C57BL/6 from Jackson Labs) intranasally (i.n.) challenged with a lethal dose of Klebisella pneumoniae strain 43816 (1×10⁵ cfu) and treated with chemokine biologic (CD0). Untreated mice serve as a control. The biologic (10 μg in 20 μl of PBS buffer) was administered via the intranasal route 1 hour before the challenge. All control (untreated) mice die by day 4, whereas a significant number of biologic-treated mice survive. Data were analyzed using log-ranked Mantel Cox test. *p<0.05.

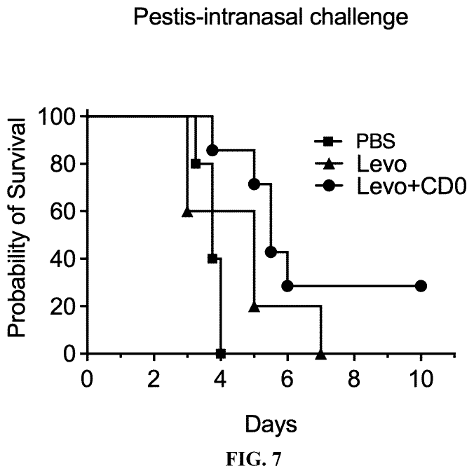

FIG. 7 is a graph showing a survival plot of 7 to 9 weeks old Swiss Albino mice (n=5) intranasally (i.n.) challenged with a lethal dose of Yersinia Pestis strain CO92 luc2 (12 LD50) and treated with chemokine biologic (CD0). The biologic (10 μg in 20 μl of PBS buffer) was administered via the intranasal route 1 hour before the challenge. All control (PBS) mice die by day 4. In this experiment, the biologic by itself is not protective and the antibiotic levofloxin imparts some protection. However, the CD0 biologic synergistically provides better protection in combination with the antibiotic levofloxin compared to antibiotic alone. Data were analyzed using log-ranked Mantel Cox test. *p<0.01 (compared to untreated mice).

As the dimer binds GAGs with higher affinity, the inventors generated Cxcl1 biologics on a disulfide-locked dimer background. The inventors designate this variant as chemokine biologico (CD0). This biologic provided protection in mice challenged with a lethal dose of intraperitoneal Salmonella Typhimurium challenge (FIG. 4); intraperitoneal or intranasal of challenge Klebisella pneumoniae (FIGS. 5, 6); and intranasal Yersinia Pestis challenge (FIG. 7).

In order to modulate GAG interactions, the biologics had an additional mutation of the N-terminal arginine to alanine (A1a) that inactivates receptor function (FIG. 3). The inventors have shown that N-terminal Arg to A1a mutants are inactive and do not recruit neutrophils in mice (9). The inventors designate this variant as chemokine biologicl (CD1). Mutations for enhancing GAG affinities were generated on this doubly modified template.

The pKa of the surface Lys amino and Arg guanidinium groups is >10 indicating that they are always protonated and charged at physiological pH. However, their binding interactions differ due to differences in their side chain structure, H-bonding, charge density, solvation, and dynamics (10). Whereas Lys amino NH3 group is symmetric and has a simple structure, the guanidinium group of Arg is planar and asymmetric with a more complex structure. Conjugation between the double bond and the nitrogen lone pairs in the guanidinium group results in delocalization of the positive charge, allowing multiple ion pair and H-bonding interactions with sulfate/carboxylate groups that are less likely for the Lys NH3 group. The guanidinium group, compared to the NH3 cation of the lysine and imidazole group of the histidine, can also form stronger electrostatic interactions with the sulfate anion based on Pearson's concept of soft acid soft base interactions (17).

The inventors expressed four single arginine mutants of the mouse Cxcl1 biologic and have characterized the structural basis of their GAG interactions. MD studies show that the newly introduced arginines are involved in stronger H-bonding interactions than the parent residue in all the four mutants (data not shown). Compared to the wild-type (WT), the MD profiles for the N-loop mutants are strikingly different whereas changes for the C-helix mutants are more subtle, indicating the location of the newly introduced arginine will differentially impact the binding interface. Affinity measurements show N-loop Arg mutants bind with ˜13 to 20-fold higher affinity, indicating that enhanced N-loop interactions compensate for any loss in C-helical interactions. Considering C-helical residues provided most stability in the WT, these data demonstrate that binding occurs in a dynamic and adaptive manner.

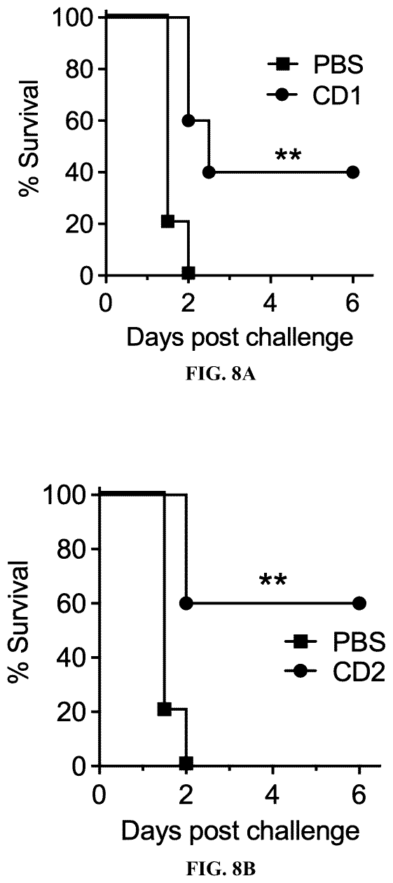

FIGS. 8A and 8B are graphs showing survival plots of 7 to 9 weeks old (n=5) mice (C57BL/6 from Jackson Labs) intraperitoneally (i.p.) challenged with a lethal LPS dose from E. coli 0111.B4 and treated with chemokine biologic CD1 (FIG. 8A) and CD2 (FIG. 8B), respectively. The biologic (10 μg in 100 μl of PBS buffer) was administered via the intraperitoneal route 1 hour before the challenge. All untreated mice serve as a control and die by day 2, whereas a significant number of biologic-treated mice survive. Data were analyzed using log-ranked Mantel Cox test. **p<0.01.

Efficacy data for biologics CD1 and H19R mutant (labeled as CD2) that shows 13-fold higher affinity than CD1 is provided herein. The biologics were administered an hour before a lethal LPS challenge in an endotoxemia disease model. The inventors chose a lethal dose for the preliminary study, as any survival can be unambiguously attributed to the biologic. Whereas untreated mice succumb within 48 hours, both CD1 and CD2 treated mice show significant survival (FIGS. 8A and 8B).

Further, a larger percent of CD2-treated compared to CD1-treated mice survived. Protection imparted by the CD2 biologic was evident as early as 8 hours post-LPS challenge, as these mice were more active compared to the untreated and CD1-treated mice. The CD2-treated mice steadily lose weight up to day 3 but regained the weight by day 6, and importantly, the extent of weight loss in these mice was less compared to that observed for the CD1-treated mice (not shown). The inventors also observed that administering the biologics at 6 hours post-LPS challenge, CD2-treated compared to CD1-treated mice showed significant survival (not shown). These data collectively provide compelling evidence that the biologics dampen neutrophil's hyperinflammatory response.

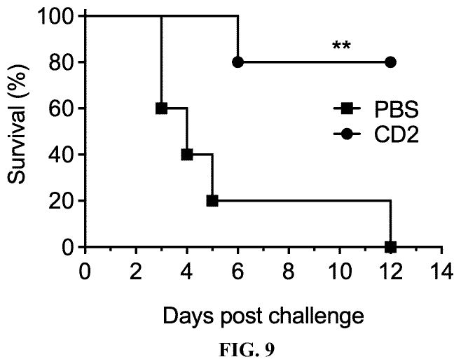

FIG. 9 is a graph showing a survival plot of 7 to 9 weeks old (n=5) mice (C57BL/6 from Jackson Labs) with Clostrodium difficile strain VPI 10463 (1×10⁴ spores) and treated with chemokine biologic CD2 6 hours before the challenge. All control (PBS) mice die by day 12, whereas a significant number of biologic-treated mice survive. Data were analyzed using log-ranked Mantel Cox test. **p<0.01, *p<0.05.

The biologics were also tested in a lethal Citrobacter colitis challenge model. Whereas the CD2 biologic conferred significant protection (FIG. 9), the CD0 biologic had no effect, indicating that selective modulation of GAG interactions is critical for the biologics to effectively alleviate disease symptoms.

### Design and Characterization of Chemokine Dimer.

The chemokine biologics were generated on the mouse chemokine Cxcl1 (also called KC or mCxcl1) background as the success of the strategy was tested in mouse disease models.

### Design of Mouse Chemokine Biologic 0 (CD0).

As the dimer binds GAGs with higher affinity, the first biologic designed by the inventors was a disulfide-locked dimer. The inventors introduced a cysteine for a residue at the two-fold symmetry (Lys27) and observed that the mouse Cxcl1 spontaneously forms a stable dimer (5). The side chain in the native protein is solvent-exposed and not involved in dimer interactions, and its backbone amide is H-bonded to the carbonyl of the adjacent β-strand within the monomer. NMR studies show that the protein is well-folded and that the disulfide is accommodated without imposing any constrains at the dimer interface (6, 7). This biologic compared to the monomer binds GAGs with higher affinity but is a weak receptor agonist (7, 8).

### Design of Mouse Chemokine Biologic 1 (CD1).

The biologic CD1 carries an additional mutation of the N-terminal arginine to alanine (A1a) that inactivates receptor function. The inventors have shown that N-terminal Arg to A1a mutants are inactive and do not recruit neutrophils in mice.

### Design of Mouse Chemokine Biologic 2 (CD2).

Mutations for enhancing GAG affinities were generated on this doubly modified template. For instance, biologic CD2 carries the H19R mutant. The inventors show that this mutant binds GAGs with much higher affinity compared to CD1.

```
mCxc11
SEQ ID NO: 11
APIANELRCQ CLQTM-AGIH LKNIQSLKVL PSGPHCTQTE VIATLKNGRE ACLDPEAPLV
                10 20 30 40 50 60
QKIVQKMLKG VPK
                70
hCXCL8
SEQ ID NO: 12
SAKELRCQCI KTYSKPFHPK FIKELRVIES GPHCANTEII VKLSDGRELC LDPKENWVQR
                10 20 30 40 50 60
VVEKFLKRAE NS
                70
```

Translation of mutations from mouse sequences to human sequences. Human CXCL8 and mouse Cxcl1 amino acid sequences (shown above) highlighting residues that are mutated to generate the different biologics. The residue highlighted in underline is mutated to generate the CD0 biologic. The residue highlighted in bold are mutated for generating the CD1 biologic on the CD0 background. Residues highlighted in italics will be mutated to generated CD2 type biologics on CD1 background. The mouse biologics start with the letter “M” (MCD0, MCD1, MCD2) and the human biologics with the letter “H” (HCD0, HCD1, HCD2). The sequences of these biologics are shown below.

```
MCD0
SEQ ID NO: 1
APIANELX2CQ CLOTM-AGIX1L X1NIQSLCVL PSGPHCTQTE VIATLKNGRE ACLDPEAPLV
                10 20 30 40 50 60
QKIVQX1MLKG VPK
                70
HCD0
SEQ ID NO: 2
SAKELX2CQCI KTYSKPFX1PX1 FIKELCVIES GPHCANTEII VKLSDGRELC LDPKENWVQR
                10 20 30 40 50 60
VVEX1FLKRAE NS
                70
X2 is an Arg, and X1 is a His or a Lys
MCD1
SEQ ID NO: 3
APIANELACQ CLQTM-AGIX1L X1NIQSLCVL PSGPHCTQTE VIATLKNGRE ACLDPEAPLV
                10 20 30 40 50 60
QKIVQX1MLKG VPK
                70
HCD1
SEQ ID NO: 4
SAKELACQCI KTYSKPFX1PX1 FIKELCVIES GPHCANTEII VKLSDGRELC LDPKENWVQR
                10 20 30 40 50 60
VVEX1FLKRAE NS
                70
X1 is an His or a Lys
MCD2
SEQ ID NO: 5
APIANELACQ CLQTM-AGIR LX1NIQSLCVL PSGPHCTQTE VIATLKNGRE ACLDPEAPLV
                10 20 30 40 50 60
QKIVQX1MLKG VPK
                70
HCD2
SEQ ID NO: 6
SAKELACQCI KTYSKPFRPX1 FIKELCVIES GPHCANTEII VKLSDGRELC LDPKENWVQR
                10 20 30 40 50 60
VVEX1FLKRAE NS
                70
X1 is a Lys
MCD2
SEQ ID NO: 7
APIANELACQ CLQTM-AGIX1 LRNIQSLCVL PSGPHCTQTE VIATLKNGRE ACLDPEAPLV
                10 20 30 40 50 60
QKIVQX1MLKG VPK
                70
HCD2
SEQ ID NO: 8
SAKELACQCI KTYSKPFX1PR FIKELCVIES GPHCANTEII VKLSDGRELC LDPKENWVQR
                10 20 30 40 50 60
VVEX1FLKRAE NS
                70
MCD2
SEQ ID NO: 9
APIANELACQ CLQTM-AGIX1 LX1NIQSLCVL PSGPHCTQTE VIATLKNGRE ACLDPEAPLV
                10 20 30 40 50 60
QKIVQRMLKG VPK
                70
HCD2
SEQ ID NO: 10
SAKELACQCI KTYSKPFX1PX1 FIKELCVIES GPHCANTEII VKLSDGRELC LDPKENWVQR
                10 20 30 40 50 60
VVERFLKRAE NS
                70
X1 is an His or a Lys
```

Cloning, expression, and purification of chemokine biologics. PCR amplification for each of the mutations was carried out using primers in which the codon of the residue at the residue to be replaced with the residue of interest using the QuikChange Site-Directed Mutagenesis kit (Agilent Technologies, CA, USA). The chemokine variant was cloned in a pET32 Xa vector and expressed and purified as a His-tag thioredoxin fusion protein. Briefly, transformed E. coli BL21 (DE3) cells will be induced with IPTG overnight at 25° C., the fusion protein cleaved with factor Xa, and purified using Ni-affinity column and HPLC. Analytical ultracentrifugation, electrophoresis, and NMR data were used to confirm that the newly introduced mutations does not perturb the native structure

All biologics that will be generated for human chemokines, and in particular CXCL8, will use the same strategy. The inventors have already expressed and charactered the HCD0 (trapped dimer) and HCD1 (inactive trapped dimer) (5, 6), and introducing mutations that enhance GAG interactions (7). The inventors have also expressed and characterized several other human chemokines as a trapped dimer for their receptor activity and GAG interactions (8-10).

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1) Kolaczkowska, E. and Kubes, P. Neutrophil recruitment and function in health and inflammation. Nat Rev Immunol 13:159-175 (2013).
2) Kruger, P., Saffarzadeh, M., Weber, A. N., et al. Neutrophils: Between host defence, immune modulation, and tissue injury. PLOS Pathog 11: e1004651 (2015).
3) Rajarathnam, K., Schnoor M., Richardson, R. M., and Rajagopal, S. How do chemokines navigate neutrophils to the target site: dissecting the signaling pathways. Cell Signal 54:69-80 (2019).
4) Rajarathnam, K. and Desai, U. R. Structural insights into how proteoglycans determine chemokine-CXCR1/CXCR2 interactions: Progress and challenges. Front Immunol 11:660 (2020).
5) Poluri, K. M., Joseph, P. R., Sawant, K. V., and Rajarathnam, K. Structural Basis of Glycosaminoglycan Binding to Chemokine CXCL1 Dimer J. Biol. Chem 288:25143-25153 (2013).
6) Sepuru, K. M., Nagarajan, B., Desai, U. R., and Rajarathnam, K. Structural basis, stoichiometry, and thermodynamics of chemokines KC/mCXCL1 and MIP2/mCXCL2 binding to glycosaminoglycan heparin J Biol Chem 293:17817-17828 (2018).
7) Sawant, K. V., Sepuru, K. M., Lowry, E., Penaranda, E., Garofalo, R., and Rajarathnam, K. Neutrophil recruitment profiles of chemokines Cxcl1/KC and Cxcl2/MIP2: Role of Cxcr2 activation and glycosaminoglycan interactions J. Leuko. Biol 109:777-791 (2021).
8) Sawant, K. V., Sepuru, K. M., Penaranda, E., Lowry, E., Garofalo, R., and Rajarathnam, K. Cxcl1-Cxcl2 heterodimer is a potent neutrophil chemoattractant J. Leuko. Biol 114:666-671 (2023).
9) Das, S. T., Rajagopalan, L., Guerrero-Plata, A., Sai, J., Richmond, A., Garofalo, R. P., and Rajarathnam, K. Monomeric and Dimeric CXCL8 are both essential for in vivo neutrophil recruitment. PLOS One 5: e11754 (2010).
10) Joseph, P. R., Sawant, K., Iwahara, J., Desai, U. R., Garofalo, R., and Rajarathnam, K. Lysines and Arginines play non-redundant roles in mediating chemokine-glycosaminoglycan interactions Sci Reports 8:12289 (2018).
11) Gangavarupu, P., Rajagopalan, L., Kolli, D., Guerrero-Plata, A., Garofalo, R. P., and Rajarathnam, K. The monomer-dimer equilibrium and glycosaminoglycan interactions of chemokine CXCL8 regulate tissue-specific neutrophil recruitment. J. Leuko. Biol. 91:259-265 (2012).
12) Sawant, K. V., Poluri, K. M., Dutta, A., Sepuru, K. M., Troshkina, A., Garofalo, R., and Rajarathnam, K. Chemokine CXCL1 mediated neutrophil recruitment: Role of glycosaminoglycan interactions. Sci. Reports 6:33123 (2016).
13) Ravindran, A., Sawant, K., Sarmiento, J. M., R. P., Navarro, J., and Rajarathnam, K. Chemokine CXCL1 dimer is a potent agonist for the CXCR2 receptor J. Biol. Chem. 288:12244-12252 (2013).
14) Brown, A. J., Sepuru, K. M., Sawant, K. V., and Rajarathnam, K. Platelet-derived Chemokine CXCL7 dimer preferentially exists in the Glycosaminoglycan-bound form: Implications for Neutrophil-Platelet crosstalk Front Immunol 8:1248 (2017).
15) Joseph, P. R., Mosier, P., Desai, P. R., and Rajarathnam, K. Solution NMR characterization of chemokine CXCL8 monomer and dimer binding to glycosaminoglycans: Structural plasticity mediates differential binding interactions. Biochem J 472:121-133 (2015).
16) Joseph, P. R., Sawant, K. V., and Rajarathnam, K. Heparin-bound chemokine CXCL8 monomer and dimer are impaired for CXCR1 and CXCR2 activation: implications for gradients and neutrophil trafficking. Open Biology 7:170168 (2017).
17) Pearson, R. G. Hard and Soft Acids and Bases. J Am Chem Soc 85:3533-3539 (1963).

What is claimed is:

1. A method for treating one or more symptoms of dysregulated inflammation in an organ of a subject in need thereof, the method comprising:
administering to the subject a composition comprising a chemokine biologic that has a higher affinity for glycosaminoglycans (GAGs) compared to wild-type chemokine and that modulates interactions between native chemokine and GAGs in an amount effective to treat one or more symptoms of dysregulated inflammation, and a pharmaceutically acceptable carrier.

2. The method of claim 1, wherein the dysregulated inflammation is systemic inflammation involving sepsis or septic shock, multiple organs, tissues, or cell types.

3. The method of claim 1, wherein the chemokine biologic forms a locked dimer.

4. The method of claim 1, wherein the chemokine biologic is selected from at least one of SEQ ID NOS: 1-10.

5. The method of claim 1, wherein the dysregulated inflammation is caused by inflammatory bowel disease, peritonitis, bacterial infection, and cancer.

6. The method of claim 5, wherein the bacterial infection is a Salmonella, Klebsiella, Yersinia, or Clostridium.

7. The method of claim 1, wherein the dysregulated inflammation is characterized by cytokine storm.

8. The method of claim 1, wherein the dysregulated inflammation is associated with acute lung injury (ALI) and/or acute respiratory distress syndrome (ARDS) associated with or arising from ventilator use, viral infection, sepsis, or systemic bacterial infections.

9. The method of claim 1, wherein the chemokine biologic reduces or corrects neutrophil hyperinflammatory or impaired response.

10. The method of claim 1, wherein the chemokine biologic is a decoy and the decoy reduces the interaction between native chemokine and GAGs.

11. The method of claim 1, wherein the composition is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration.

12. The method of claim 1, wherein the composition is administered prior to, in conjunction, subsequent to, or in alternation with treatment with one or more additional therapies or procedures selected from the group consisting of antimicrobials, surfactant, and corticosteroids.

13. The method of claim 1, wherein the composition is in an amount between about 0.1 and about 40 mg/kg body weight of the subject.

14. The method of claim 1, wherein the composition is in an amount between about 2 and about 8 mg/kg body weight of the subject, and wherein the composition is administered by subcutaneous injection.

15. The method of claim 1, wherein the composition is administered in an amount effective to reduce at least one of:
one or more pro-inflammatory chemokines selected from the group consisting of CXCL8, CXCL1, CXCL2, CXCL3, CXCL5, CXCL6, and CXCL7; or
one or more pro-inflammatory cytokines selected from IL-6, TNF-α, IL-12, IL-1a, and IL-18, and inflammation related proteins C-reactive protein and ferritin.

16. The method of claim 1, wherein one or more pro-inflammatory cells are neutrophils.

17. A peptide comprising a chemokine biologic that has a higher affinity for glycosaminoglycans compared to wild-type chemokines and that prevents or reduces native chemokine binding glycosaminoglycans (GAGs) in an amount effective to treat one or more symptoms of dysregulated inflammation.

18. The peptide of claim 17, wherein the chemokine biologic forms a locked dimer.

19. The peptide of claim 17, wherein the chemokine biologic is a chemokine variant is selected from at least one of SEQ ID NOS: 1-10.

20. The peptide of claim 17, further comprising a pharmaceutically acceptable carrier.

21. The peptide of claim 17, wherein the chemokine biologic is formulated for intravenous, oral, enteral, parenteral, topical, intramuscular, subcutaneous, intradermal, transdermal, rectal, vaginal, mucosal, buccal, intraperitoneal, intranasal, sublingual, intranasal, intrapulmonary administration.

22. The peptide of claim 17, wherein the chemokine biologic is in an amount between about 0.1 and about 40 mg/kg body weight.

23. The peptide of claim 17, wherein the chemokine biologic is in an amount between about 2 and about 8 mg/kg body weight, and wherein the chemokine biologic is administered by subcutaneous injection.

24. A method for treating one or more symptoms of dysregulated inflammation in an organ of a subject in need thereof, the method comprising:
identifying a subject in need of treatment for a dysregulated inflammation triggered by neutrophils; and administering to the subject a composition comprising a chemokine biologic that has a higher affinity for glycosaminoglycans (GAGs) compared to wild-type chemokines and that prevents or reduces native chemokine binding to GAGs in an amount effective to treat one or more symptoms of dysregulated inflammation.

25. A method for treating one or more symptoms of dysregulated inflammation in an organ of a subject in need thereof, the method comprising: administering to the subject a chemokine biologic that has a higher affinity for glycosaminoglycans (GAGs) compared to wild-type chemokine and that prevents or reduces native chemokine binding to GAGs in an amount effective to treat one or more symptoms of dysregulated inflammation, and a pharmaceutically acceptable carrier.

26. The method of claim 25, wherein the chemokine biologic is administered in an amount effective to reduce at least one of:
one or more pro-inflammatory chemokines selected from the group consisting of CXCL8, CXCL1, CXCL2, CXCL3, CXCL5, CXCL6, and CXCL7; or
one or more pro-inflammatory cytokines selected from IL-6, TNF-α, IL-12, IL-1α, and IL-18, and inflammation related proteins C-reactive protein and ferritin.

27. The method of claim 25, wherein one or more pro-inflammatory cells are neutrophils.
